SEQUENCE LISTING (19 sequences)

SEQ ID NO: 1 - protein, 17 aa, synthetic construct
  REGION 1..17: note = Synthetic peptide
MYSFVSEETGTLIVNSV

SEQ ID NO: 2 - protein, 15 aa, synthetic construct
  REGION 1..15: note = Synthetic peptide
LLHAPATVCGPKKST

SEQ ID NO: 3 - protein, 15 aa, synthetic construct
  REGION 1..15: note = Synthetic peptide
NKKFLPFQQFGRDIA

SEQ ID NO: 4 - protein, 15 aa, synthetic construct
  REGION 1..15: note = Synthetic peptide
PHGVVFLHVTYVPAQ

SEQ ID NO: 5 - protein, 76 aa, unidentified
  REGION 1..76: note = SARS-CoV-2 envelope protein
MYSFVSEETGTLIVNSVLLFFAFVVFLLVTLAILTALRLCAYCCNIVNVSLVKPTVYVYSRVKNLNSSEGVPDLLV

SEQ ID NO: 6 - protein, 1297 aa, unidentified
  REGION 1..1297: note = SARS-CoV-2 spike protein
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQNVNCTEVPVAIHADQLTPTWRVYSTGSNVFQTRAGCLIGAEHVNNSYECDIPIGAGICASYQTQTNSPSRAGSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGPALQIPFAMQMAYRFNGIGVTQNVLYENQKLIANQFNSAIGKIQDSLSSTPSALGKLQDVVNQNAQALNTLVKQLSSNFGAISSVLNDILSRLDKPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGRSLEVLFQGPGSLPETGGGSDYKDDDDKGGGGSGGGGSGGGGSGGGGSGGGGSHHHHHH

SEQ ID NO: 7 - protein, 8 aa, synthetic construct
  REGION 1..8: note = Synthetic peptide
MYSYVTEE

SEQ ID NO: 8 - protein, 7 aa, synthetic construct
  REGION 1..7: note = Synthetic peptide
YSFVTEE

SEQ ID NO: 9 - protein, 6 aa, synthetic construct
  REGION 1..6: note = Synthetic peptide
VSEETG

SEQ ID NO: 10 - protein, 11 aa, synthetic construct
  REGION 1..11: note = Synthetic peptide
YSFRSVREETG

SEQ ID NO: 11 - protein, 8 aa, synthetic construct
  REGION 1..8: note = Synthetic peptide
MYAFRSEE

SEQ ID NO: 12 - protein, 10 aa, synthetic construct
  REGION 1..10: note = Synthetic peptide
YSFFSEEGTG

SEQ ID NO: 13 - protein, 10 aa, synthetic construct
  REGION 1..10: note = Synthetic peptide
LHCPADVCGP

SEQ ID NO: 14 - protein, 9 aa, synthetic construct
  REGION 1..9: note = Synthetic peptide
LLHEHATVC

SEQ ID NO: 15 - protein, 16 aa, synthetic construct
  REGION 1..16: note = Synthetic peptide
LSEANTKFMFDLFQQF

SEQ ID NO: 16 - protein, 12 aa, synthetic construct
  REGION 1..12: note = Synthetic peptide
LTESNKSVLQFQ

SEQ ID NO: 17 - protein, 9 aa, synthetic construct
  REGION 1..9: note = Synthetic peptide
GVVFPHVSY

SEQ ID NO: 18 - protein, 11 aa, synthetic construct
  REGION 1..11: note = Synthetic peptide
FLYVHHTYVPA

SEQ ID NO: 19 - protein, 7 aa, synthetic construct
  REGION 1..7: note = Synthetic peptide
LNVTYVP

(19) United States
(12) Patent Application Publication
(10) Pub. No.: US 2025/0044280 A1
(43) Pub. Date: Feb. 6, 2025

(54) METHODS FOR DIAGNOSING OR PREDICTING A CARDIAC INFLAMMATORY CONDITION

(71) Applicants: QUEEN MARY UNIVERSITY OF LONDON, London (GB); BARTS HEALTH NHS TRUST, London (GB)

(72) Inventors: Federica Maria MARELLI-BERG, London (GB); Maria Paula LONGHI, London (GB); Saidi MOHIDDIN, London (GB)

(73) Assignees: QUEEN MARY UNIVERSITY OF LONDON, London (GB); BARTS HEALTH NHS TRUST, London (GB)

(21) Appl. No.: 18/715,512

(22) PCT Filed: Dec. 2, 2022
(86) PCT No.: PCT/GB2022/053077

§ 371 Date: May 31, 2024

(30) Foreign Application Priority Data
Dec. 2, 2021 (GB) 2117397.6

(51) Int. Cl. G01N 33/50

(52) U.S. Cl. CPC G01N 33/505; G01N 2800/32; G01N 2800/7095

(57) ABSTRACT

The present invention relates to diagnosis of a cardiac inflammatory condition, such as myocarditis. In particular, the invention relates to a method for diagnosing a cardiac inflammatory condition in a subject suffering from, or who has been vaccinated against, COVID-19 by determining the level of cMet-positive T cells in a sample from the subject. The invention further relates to a method for predicting whether a subject will develop a cardiac inflammatory condition and a method of treating patient suffering from a cardiac inflammatory condition.

### FIELD OF THE INVENTION

The present invention relates to diagnosis of a cardiac inflammatory condition, such as myocarditis. In particular, the invention relates to a method for diagnosing a cardiac inflammatory condition in a subject suffering from, or who has been vaccinated against, COVID-19 by determining the level of cMet-positive T cells in a sample from the subject. The invention further relates to a method for predicting whether a subject will develop a cardiac inflammatory condition and a method of treating a subject suffering from a cardiac inflammatory condition.

### BACKGROUND TO THE INVENTION

Infection with severe acute respiratory syndrome-coronavirus-2 (SARS-COV-2), the virus responsible for coronavirus disease 2019 (COVID-19), can cause cardiac inflammation and damage (Kariyanna et al. Am J Med Case Rep 2020 8 (9): 299-305). Cardiac inflammation has also been reported after vaccination with mRNA-based COVID-19 vaccines, in particular the Moderna vaccine (mRNA-1273) and Pfizer/BioNTech vaccine (BNT162b2) (Albert et al. Rad Case Rep 2021 16 (8) 2142-2145; Bautista et al. Rev Esp Cardiol (Engl Ed) 2021 74 (9) 812-814; Diaz et al. JAMA 2021 326 (12): 1210-1212).

Cardiac inflammation enlarges and weakens the heart, reducing the efficiency with which blood is pumped around the body. Chronic cardiac inflammation can damage cardiomyocytes and in severe cases lead to disabling symptoms, progressive heart failure, cardiac arrest or stroke. The most common cause of cardiac inflammation is viral infection. There are three types of cardiac inflammation: myocarditis (inflammation of the heart muscle), pericarditis (inflammation of the outer lining of the heart) and endocarditis (inflammation of the inner lining of the heart). Myocarditis and pericarditis have been associated with both COVID-19 and vaccination against COVID-19. The COVID-19 virus may infect heart cells via interaction of its spike protein with angiotensin-converting enzyme 2 receptors (ACE2), directly causing cardiac damage and inflammation (Aleksova et al. In J Mol Sci 2021, 22, 4526). Alternatively or additionally, the COVID-19 virus may trigger a general hyperimmune response, thereby indirectly causing cardiac inflammation (Castiello et al. Heart Fail Rev 2021 1-11). Similarly, a COVID-19 vaccine may induce a hyperimmune response, leading to cardiac inflammation.

The incidence rate of myocarditis from infection with the COVID-19 virus has been reported as 11 events per 100,000 persons, whilst the risk of myocarditis from vaccination with mRNA-based COVID-19 vaccine is lower, at 2.7 events per 100,000 persons (Barda et al. N Engl J Med 2021 385:1078-90). Incidence rates of myocarditis post-vaccine are highest after a second dose of vaccine in young males between 16 and 19 years of age, and onset occurs within 21 days of first dose or 30 days of second dose (Mevorach et al. 2021 DOI: 10.1056/NEJMoa2109730).

Cardiac inflammation is mediated by the immune system, including effector T lymphocytes (effector T cells). Effector T cells migrate to specific sites according to the set of adhesion molecules and chemokine receptors (“homing receptors”) they express. Migration of effector T cells to the heart (“cardiotropism”) is mediated by the hepatocyte growth factor (HGF) receptor cMet. Specifically, T cells primed in heart-draining lymph nodes in the presence of HGF produced in the cardiac parenchyma during inflammation are induced to express a molecular code that promotes their recirculation through the heart. In both mice and humans, cardiotropic T cells are characterized by expression of cMet and chemokine receptors CXCR3 and CCR4 (Komarowska et al. Immunity 2015 42, 1087-1099).

Existing methods to diagnose cardiac inflammation include electrocardiogram (detecting the pattern of electrical activity of the heart), echocardiogram (detecting sounds waves reflected from the beating heart), chest X-ray (to assess heart morphology and presence of fluid in or around the heart) and cardiac MRI (to assess heart function and morphology, and myocardial tissue characteristics). In more severe cases, cardiac catheterisation and heart muscle biopsy may be performed. Blood tests are also used to check for proteins typically elevated in cardiac inflammation due to cardiac damage, such as cardiac troponin I (cTnI), cardiac troponin T (cTnT), b-type natriuretic peptide (BNP) and N-terminal proBNP (NT-proBNP). Such methods have been used to diagnose cardiac inflammation in subjects suffering from COVID-19 (Siripanthong et al. Heart Rhythm 2020 17 (9): 1463-1471).

The present invention provides an alternative blood biomarker, namely cMet-positive T cells, for diagnosing cardiac inflammation specifically in subjects suffering from, or vaccinated against, COVID-19.

### SUMMARY OF THE INVENTION

The present inventors have identified that cardiotropic, cMet-positive T cells are increased in the blood of animal subjects immunised with peptides derived from COVID-19 virus spike protein and envelope protein which have similar sequences to parts of the sequence of cardiac proteins. The subjects exhibit hallmarks of myocarditis, such as cardiac dysfunction. Without wishing to be bound by theory, it is hypothesised that COVID-19 virus spike protein and envelope protein in an infected subject or subject who has been vaccinated against COVID-19 induce an immune response which inadvertently also targets cardiac self-antigens, leading to cardiac inflammation. Cardiac inflammation is mediated by cardiotropic, cMet-positive T cells. Accordingly, the level of cMet-positive T cells in a subject suffering from COVID-19, or who has been recently vaccinated against COVID-19, may be used as a biomarker for diagnosis of a cardiac inflammatory condition. Furthermore, the level of such cMet-positive T cells in a subject may also be used to predict that the subject will develop a cardiac inflammatory condition before other signs of cardiac inflammation become evident.

Generally, the invention relates to methods for diagnosing or predicting a cardiac inflammatory condition in a subject suffering from, or who has been vaccinated against, COVID-19.

The invention provides a method for diagnosing a cardiac inflammatory condition in a subject, wherein the subject
(i) is suffering from COVID-19, and/or
(ii) has been vaccinated against COVID-19 within 30 days prior to carrying out said method of diagnosis,
   the method comprising the steps of:
(a) determining a level of cMet-positive T cells in a sample obtained from the subject; and
(b) comparing the level of cMet-positive T cells in the sample to a reference value, wherein the level of cMet-positive T cells in the sample compared to the reference value is indicative of the presence or absence of the cardiac inflammatory condition in the subject.

The invention also provides a method for predicting whether a subject will develop a cardiac inflammatory condition, wherein the subject
(i) is suffering from COVID-19, and/or
(ii) has been vaccinated against COVID-19 within 30 days prior to carrying out said method of prediction,
   the method comprising the steps of:
(a) determining a level of cMet-positive T cells in a sample obtained from the subject; and
(b) comparing the level of cMet-positive T cells in the sample to a reference value, wherein the level of cMet-positive T cells in the sample compared to the reference value is predictive of whether the subject will develop the cardiac inflammatory condition.

The invention also provides a method of treating a subject suffering from a cardiac inflammatory condition, the method comprising the steps of:
(a) diagnosing a cardiac inflammatory condition using the method for diagnosing a cardiac inflammatory condition of the invention; and
(b) administering to the patient an effective amount of a treatment, therapy or prophylaxis for a cardiac inflammatory condition.

The invention also provides an effective amount of a treatment, therapy or prophylaxis for a cardiac inflammatory condition for use in a method of treating a subject suffering from a cardiac inflammatory condition, wherein the patient has been diagnosed with a cardiac inflammatory condition using the method for diagnosing a cardiac inflammatory condition of the invention.

A principal advantage of the present invention is that the method of diagnosis detects a component of the immune response that mediates cardiac inflammation, namely cMet-positive T cells. In contrast, methods of diagnosing cardiac inflammation known in the art detect cardiac dysfunction (e.g. via X-ray, electrocardiogram or echocardiogram) or cardiac damage (e.g. through blood tests for factors produced by damaged heart tissue, such as troponin) that result from cardiac inflammation. Thus, for successful diagnosis via the method of the present invention, cardiac inflammation is not required to have progressed to a stage where cardiac dysfunction is evident or actual damage to the heart has occurred. Accordingly, one aspect of the present invention is a method of predicting that a subject will develop a cardiac inflammatory condition. In this respect, cMet-positive T cells may be detected, and thus a cardiac inflammatory condition predicted, before other symptoms of the cardiac inflammatory condition manifest.

As cMet-positive T cells are detected in peripheral blood, the diagnostic method of the invention also has the advantage that it can be carried out conveniently alongside and in addition to existing blood tests for diagnosing a cardiac inflammatory condition, such as detection of troponin and BNP. Additionally, in contrast to catheterisation and heart biopsy, the diagnostic method of the invention does not require invasive intervention on the subject.

### DESCRIPTION OF THE FIGURES

FIG. 1—Levels of circulating CD3+CD4+ cMet+ T cells (A) and CD3+CD8+ cMet+ T cells (B), heart weight to body weight ratio (C) and ejection fraction (E) in mice immunised subcutaneously with a cocktail of four COVID peptides (SEQ ID NOs 1-4) and the indicated adjuvant—none (control), alum+MPLA, R848, AS03+MPLA. Haematoxylin and eosin staining of a heart from a control mouse and a heart from a mouse immunised with peptide cocktail including AS03+MPLA adjuvant (D).

FIG. 2—Proliferation of CD4+ cMet+ T cells upon immunisation of mice with peptide cocktail, with or without AS03+MPLA adjuvant (stimulated), or unstimulated.

FIG. 3—Levels of circulating CD4+ cMet+ T cells and CD8+ cMet+ T cells (A) and echocardiography data (B) from mice immunised intranasally with a cocktail of four COVID peptides (SEQ ID NOs 1-4) and the AS03+MPLA adjuvant (control mice immunised with AS03+MPLA adjuvant alone). Haematoxylin and eosin staining of hearts from two mice immunised with peptide cocktail including AS03+MPLA adjuvant (C).

FIG. 4—Levels of circulating CD3+CD4+ cMet+ T cells and CD3+CD8+ cMet+ T cells in mice immunised with individual COVID peptides in AS03+MPLA adjuvant (control mice immunised with AS03+MPLA adjuvant alone).

FIG. 5—Gating strategy from data for single cells (A) and lymphocytes identified within the blood (B). Granulocyte and monocyte populations also seen.

### DETAILED DESCRIPTION

### Cardiac Inflammatory Condition

The invention provides a method for diagnosing a cardiac inflammatory condition in a subject, and a method for predicting whether a subject will develop a cardiac inflammatory condition. The invention also provides a method of treating a subject suffering from a cardiac inflammatory condition and an effective amount of a treatment, therapy or prophylaxis for a cardiac inflammatory condition for use in a method of treating a patient suffering from a cardiac inflammatory condition.

A cardiac inflammatory condition is inflammation of the heart. In some embodiments, the cardiac inflammatory condition is myocarditis. Myocarditis is inflammation of the myocardium (heart muscle), which can cause arrhythmia of the heart and reduce the ability of the heart to pump blood. Symptoms of myocarditis include chest pain, abdominal pain, rapid or irregular heartbeat, fainting, fatigue, shortness of breath and general weakness, fever and swelling of legs or feet.

In some embodiments, the cardiac inflammatory condition is pericarditis. Pericarditis is inflammation of the pericardium, which is a fluid-filled sac around the heart. Symptoms of pericarditis include chest pain, fever shortness of breath and a fast heartbeat.

In some embodiments, the cardiac inflammatory condition encompasses both myocarditis and pericarditis. In other words, the method of the invention may diagnose the subject as having, or predict that the subject will develop, both myocarditis and pericarditis.

In some embodiments, the cardiac inflammatory condition is endocarditis. Endocarditis is inflammation of the inner lining of the chambers and valves of the heart. Symptoms of endocarditis include chest pain, abdominal pain, shortness of breath, cough, fever, blood in urine, muscle, joint and back pain, night sweats and skin changes. In some embodiments, the cardiac inflammatory condition encompasses myocarditis, pericarditis and endocarditis.

### COVID-19

The invention provides a method for diagnosing a cardiac inflammatory condition in a subject, and a method for predicting whether a subject will develop a cardiac inflammatory condition, wherein the subject is infected with COVID-19 virus and/or suffering from one or more morbidity associated with COVID-19.

The term “COVID-19” refers to the disease caused by infection with SARS-COV-2. The terms “COVID-19 virus” and “SARS-COV-2” are used interchangeably herein. The genome of SARS-CoV-2 has been sequenced (Wu et al. Nature 2020 579, 265-269), and changes in the genome have been reported (Tang et al. Nat Sci Rev 7 (6): 1012-1023). SARS-COV-2 is a coronavirus with a single-stranded positive sense RNA genome of about 30,000 nucleotides in length. The virus genome encodes four structural proteins: the spike surface glycoprotein(S), small envelope protein (E), matrix protein (M) and nucleocapsid protein (N). The spike protein binds receptors on the host cell surface and mediates membrane fusion (Wrapp et al. Science 2020 367 (6483): 1260-1263). The envelope, matrix and nucleocapsid proteins function in encasing the RNA genome and mediating virus assembly, budding and envelope formation. Full details of the molecular biology, life cycle, mechanism of infection and associated pathology of SARS-COV-2 are readily available in the art. The term “COVID-19 virus” encompasses SARS-COV-2 and any natural or engineered variants thereof, including particularly natural variants that may develop in the future through mutation of SARS-COV-2.

In some embodiments, the subject is infected with COVID-19 virus. Whether a person is infected with COVID-19 virus may be readily determined using techniques known in the art (reviewed in, for example, Song et al. Lab Chip 2021 21 (9): 1634-1660), such as polymerase chain reaction (PCR) based tests to identify the presence of SARS-COV-2 genome in the subject and immunoassays to detect antibodies against SAS-COV-2. A subject infected with COVID-19 virus may exhibit viremia (presence of COVID-19 virus in the bloodstream).

In some embodiments, the subject is suffering from one or more morbidity associated with COVID-19. A morbidity associated with COVID-19 is a symptom or condition caused directly or indirectly by infection with the COVID-19 virus. Morbidities associated with COVID-19 are known in the art and may be readily recognised by a skilled medical practitioner. Examples of symptoms of COVID-19, which are considered morbidities associated with COVID-19 in the context of the present invention, include high temperature, fever or chills, cough, sore throat, shortness of breath or difficulty breathing, chest pain, fatigue, headache, loss or change to sense of smell or taste, nausea or vomiting and diarrhoea. Other morbidities associated with COVID-19 include pneumonia, acute respiratory distress, lung sepsis and cardiac inflammation, such as myocarditis or pericarditis.

In some embodiments, the subject is both infected with the COVID-19 virus and suffering from one or more morbidity associated with COVID-19. In other embodiments, the subject is not infected with the COVID-19 virus but is suffering from one or more morbidity associated with COVID-19. In such embodiments, the subject may have previously been infected with COVID-19 virus and since cleared the infection but is still suffering from one or more morbidity associated with COVID-19. Thus, in some embodiments, the subject is suffering from one or more morbidity associated with a prior COVID-19 infection in the subject. In other embodiments, the subject is infected with the COVID-19 virus but is not noticeably suffering from any morbidity associated with COVID-19. In other words, the subject may be asymptomatic.

In some embodiments of the invention, the subject is suffering from long COVID. The symptoms and conditions found in long COVID cases may be considered morbidities associated with COVID-19. In other words, in some embodiments, a subject suffering from one or more morbidity associated with COVID-19 is suffering from long COVID. Long COVID may also be referred to as “post COVID-19 condition”. Long COVID is a set of persistent conditions experienced by some subjects that have been infected with SAS-COV-2. The World Health Organisation definition states that long COVID (post COVID-19 condition) occurs in individuals with a history of probable or confirmed SARS-COV-2 infection, usually 3 months from the onset of COVID-19 with symptoms that last for at least 2 months and cannot be explained by an alternative diagnosis. Common symptoms include fatigue, shortness of breath, cognitive dysfunction but also others and generally have an impact on everyday functioning. Symptoms may be new onset following initial recovery from an acute COVID-19 episode or persist from the initial illness. Symptoms may also fluctuate or relapse over time (WHO-A clinical case definition of post COVID-19 condition by a Delphi consensus, 6 Oct. 2021).

### Vaccination Against COVID-19

The invention provides a method for diagnosing a cardiac inflammatory condition in a subject, and a method for predicting whether a subject will develop a cardiac inflammatory condition, wherein the subject has been vaccinated against COVID-19. In some embodiments, the subject is both infected with COVID-19 virus and has been vaccinated against COVID-19. In some embodiments, the subject is both suffering from one or more morbidity associated with COVID-19 and has been vaccinated against COVID-19. In some embodiments, the subject is infected with COVID-19 virus, suffering from one or more morbidity associated with COVID-19 and has been vaccinated against COVID-19.

In some embodiments, the subject has been vaccinated against COVID-19 within 6 weeks prior to carrying out the method of diagnosis or prediction. In some embodiments, the subject has been vaccinated against COVID-19 within 6 weeks prior to carrying out the method of diagnosis. In some embodiments, the subject has been vaccinated against COVID-19 within 6, 5, 4, 3, 2 or 1 weeks prior to carrying out the method of diagnosis or prediction. In some embodiments, the subject has been vaccinated against COVID-19 within 56 days prior to carrying out the method of diagnosis. In some embodiments, the subject has been vaccinated against COVID-19 within 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2 or 1 days prior to carrying out the method of diagnosis. Preferably, the subject has been vaccinated against COVID-19 within 30, 21, 14, 10 or 7 days prior to carrying out the method of diagnosis.

In some embodiments, the subject has been vaccinated against COVID-19 within 6 weeks prior to carrying out the method of prediction. In some embodiments, the subject has been vaccinated against COVID-19 within 6, 5, 4, 3, 2 or 1 weeks prior to carrying out the method of prediction. In some embodiments, the subject has been vaccinated against COVID-19 within 56 days prior to carrying out the method of prediction. In some embodiments, the subject has been vaccinated against COVID-19 within 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2 or 1 days prior to carrying out the method of prediction. Preferably, the subject has been vaccinated against COVID-19 within 30, 21, 14, 10 or 7 days prior to carrying out the method of prediction.

The expression “vaccinated against COVID-19” means that a vaccine intended to provide at least some degree of immunity against COVID-19 has been administered to the subject. Such vaccines are referred to herein as “COVID-19 vaccines”. Subjects vaccinated with any COVID-19 vaccine, including vaccines not authorised or developed or at the date of filing of the present application, are within the scope of the invention. In some embodiments, the subject has been vaccinated against COVID-19 using a vaccine selected from the following: BNT162b2 (the Pfizer/BioNTech vaccine), mRNA-1273 (the Moderna vaccine), AZD1222 (the Oxford/AstraZeneca vaccine), the Sputnik V vaccine, AD5-nCOV (Convidecia), Ad26.COV2.S (the Janssen vaccine), CoronaVac (the Sinovac vaccine), BBIBP-CorV, WIBP-CorV, Covaxin, EpiVacCorona, ZF2001 (ZIFIVAX) and MVC-COV1901.

In some embodiments, the subject has been vaccinated against COVID-19 using an mRNA vaccine. An “mRNA vaccine” is a vaccine wherein the active ingredient is a nucleoside-modified mRNA encoding a component of the COVID-19 virus. Examples of mRNA vaccines are BNT162b2 and mRNA-1273.

In some embodiments of the invention, the subject has been vaccinated against COVID-19 using BNT162b2 vaccine. Other names for BNT162b2 include “the Pfizer/BioNtech COVID-19 vaccine”, “the Pfizer/BioNtech vaccine” or “the Pfizer vaccine” (as it was developed by the companies Pfizer and BioNTech), “Comirnaty” (which is its brand name) and “tozinameran”. BNT162b2 is an mRNA vaccine as the active ingredient is a nucleoside-modified mRNA encoding the viral spike glycoprotein of the COVID-19 virus. Full details of BNT162b2 are readily available in the art (see for example Polack et al. N Engl J Med 2020 383:2603-2615).

In some embodiments, the subject has been vaccinated against COVID-19 using mRNA-1273 vaccine. Other names for mRNA-1273 include “the Moderna COVID-19 vaccine” or simply “the Moderna vaccine” (as it was developed by the company Moderna), “Spikevax” (which is its brand name), “CX-024414” and “TAK-919”. mRNA-1273 is an mRNA vaccine as the active ingredient is a nucleoside-modified mRNA encoding the viral spike glycoprotein of the COVID-19 virus. Full details of mRNA-1273 are readily available in the art (see for example Baden et al. N Engl J Med 2021 384:403-416).

Some COVID-19 vaccines are administered as a single dose (e.g. the Janssen vaccine), whilst other COVID-19 vaccines require two administrations (i.e. two doses) separated by a number of weeks or months (e.g. the Pfizer/BioNTech vaccine) to induce immunity against COVID-19. A COVID-19 vaccine may also be administered as a “booster”, meaning that the vaccine is administered to a subject who has already been administered the requisite number of doses to acquire immunity against COVID-19. A booster dose is typically administered months after the original vaccination doses. The present invention encompasses all of these types of administration. That is, in some embodiments, the vaccination of the subject against COVID-19 within 30 days prior to carrying out the method of diagnosis or prediction is the single vaccination required to confer immunity, the first vaccination of two intended vaccinations, the second vaccination of two vaccinations or a booster vaccination. Where a subject has been vaccinated against COVID-19 multiple times, the period of “within 30 days prior to carrying out the method of diagnosis/prediction” is measured from the most recent vaccination. Myocarditis has been observed to be more common after the second of two vaccinations. Thus, in some embodiments, the vaccination is the second of two vaccinations against COVID-19.

### Subject

In some embodiments, the subject is a human. The subject may be any age, gender or ethnicity. The subject may be a human adult or human child. In the context of the present invention, a “human adult” is a human subject of 16 years of age or older at the time of sampling, and a “human child” is a human subject of less than 16 years of age at the time of sampling.

In some embodiments, the subject is a male human. In some embodiments, the subject is from 10 to 60 years of age. In some embodiments, the subject is from 16 to 40 years of age. In some embodiments, the subject is from 16 to 30 years of age. In some embodiments, the subject is from 16 to 21 years of age. In some embodiments, the subject is from 16 to 19 years of age.

The subject may exhibit one or more symptoms of COVID-19. The subject may exhibit one or more symptoms of a cardiac inflammatory condition. The subject may have been previously characterised as having a cardiac inflammatory condition by other diagnostic methods. Where the results of previous tests are ambiguous or inconclusive, the method of the present invention may be used to confirm the diagnosis.

The subject may have a predisposition to develop a cardiac inflammatory condition. For example, there may be an increased risk or likelihood that the subject will develop a cardiac inflammatory condition at some point in the future.

cMet-Positive T Cells

The invention provides a method comprising the steps of:
(a) determining a level of cMet-positive T cells in a sample obtained from the subject; and
(b) comparing the level of cMet-positive T cells in the sample to a reference value, wherein the level of cMet-positive T cells in the sample compared to the reference value is indicative of the presence or absence in the subject of, or predictive of whether the subject will develop, the cardiac inflammatory condition.

cMet-positive T cells are effector T cells expressing the hepatocyte growth factor (HGF) receptor cMet. cMet is also known as MET, hepatocyte growth factor receptor (HGFR), tyrosine-protein kinase Met, AUTS9, RCCP2 and DFNB97. cMet is a single-membrane-pass receptor tyrosine kinase. Binding of the chemokine HGF to cMet triggers multiple downstream signalling pathways that may influence gene expression and thereby cellular phenotype and/or behaviour in a variety of ways depending on cell type. Binding of HGF to cMet on T cells during T cell activation induces cardiotropic behaviour (i.e. triggers migration of the T cells to the heart). cMet-positive T cells may also be denoted as cMet+ T cells.

In some embodiments, the cMet-positive T cells are cardiotropic T cells. “Cardiotropic” means that the T cells are attracted towards and function within the heart. In some embodiments, the cMet-positive T cells are virus-induced cardiotropic T cells. The term “virus-induced” means that the T cells have developed the cardiotropic phenotype in response to a virus, such as the COVID-19 virus.

### Other T Cell Markers—CCR4, CXCR3, CD4, CD8

In some embodiments, the cMet-positive T cells are CCR4-positive T cells. In other words, the T cells express both cMet and CCR4. Accordingly, in some embodiments the method of the invention comprises determining a level of cMet-positive, CCR4-positive T cells (i.e. cMet+CCR4+ T cells). CCR4 (CC chemokine receptor 4) is a receptor for two CC chemokine ligands (CCL17 and 22) that is expressed on T cells. CCR4 is also known as CKR4, CC-CKR4, K5-5, CD194, CMKBR4 and ChemR13.

In some embodiments, the cMet-positive T cells are CXCR3-positive T cells. In other words, the T cells express both cMet and CXCR3. Accordingly, in some embodiments the method of the invention comprises determining a level of cMet-positive, CXCR3-positive T cells (i.e. cMet+CXCR3+ T cells). CXCR3 (CXC receptor 3) is a receptor for three CXC chemokine ligands (CXCL9, 10 and 11) that is expressed on T cells. CXCR3 is also known as GPR9, MigR, CD182, CD183, CKR-L2, CMKAR3 and IP10-R.

In some embodiments, the cMet-positive T cells are cMet-positive, CCR4-positive, CXCR3-positive T cells (i.e. cMet+CCR4+CXCR3+ T cells). In other words, the T cells express all three of cMet, CCR4 and CXCR3. Accordingly, in some embodiments the method of the invention comprises determining a level of cMet-positive, CCR4-positive T cells, CXCR3-positive T cells (i.e. cMet+CCR4+CXCR3+ T cells). cMet+CCR4+CXCR3+ is a specific molecular signature expressed by cardiotropic T cells.

In some embodiments, the cMet-positive T cells are CD4-positive T cells. In other words, the T cells express both cMet and CD4. Accordingly, in some embodiments the method of the invention comprises determining a level of cMet-positive, CD4-positive T cells. CD4 (cluster of differentiation 4) is a co-receptor for the T cell receptor that is expressed on the surface of immune cells. CD4 is also known as IMD79, OKT4D and CD4mut.

In some embodiments, the cMet-positive T cells are CD8-positive T cells. In other words, the T cells express both cMet and CD8. Accordingly, in some embodiments the method of the invention comprises determining a level of cMet-positive, CD8-positive T cells. CD8 (cluster of differentiation 8) is a co-receptor for the T cell receptor that is expressed on the surface of immune cells. There are two isoforms of CD8, alpha and beta, denoted CD8a and CD8b respectively. “CD8” as used herein refers to both isoforms. In other words, CD8-positive T cells are T cells expressing either CD8a alone, CD8b alone or both CD8a and CD8b. CD8a is also known as p32 and Leu2. CD8b is nalos known as LY3, P37, LEU2, LYT3 and CD8B1.

All references herein to determining the level of cMet-positive T cells encompass determining the level of T cells that express cMet as well as any of CCR4, CXCR3, CD4, CD8, or any combination of these cell surface proteins. Thus, in some embodiments, determining a level of cMet-positive T cells means determining a level of cMet-positive, CCR4-positive T cells (i.e. detecting cells that express cMet and CCR4). In some embodiments, determining a level of cMet-positive T cells means determining a level of cMet-positive, CXCR3-positive T cells (i.e. detecting cells that express cMet and CXCR3). In some embodiments, determining a level of cMet-positive T cells means determining a level of cMet-positive, CCR4-positive, CXCR3-positive T cells (i.e. detecting cells that express cMet, CCR4 and CXCR3).

### Sequences

Amino acid and nucleotide sequences of human cMet, CCR4, CXCR3, CD4 and CD8 are available from publicly accessible databases, e.g. under the accession numbers as shown in the table below:

| | HGNC | Ensembl | UniProt | RefSeq (mRNA) | RefSeq (protein) |
|---|---|---|---|---|---|
| cMet | 7029 | ENSG00000105976 | P08581 | NM_000245.4 | NP_000236.2 |
| | | | | NM_001127500.3 | NP_001120972.1 |
| | | | | NM_001324401.3 | NP_001311330.1 |
| | | | | NM_001324402.2 | NP_001311331.1 |
| CCR4 | 1605 | ENSG00000183813 | P51679 | NM_005508.5 | NP_005499.1 |
| CXCR3 | 4540 | ENSG00000186810 | P49682 | NM_001142797.2 | NP_001136269.1 |
| | | | | NM_0010504.2 | NP_001495.1 |
| CD4 | 1678 | ENSG00000010610 | P01730 | NM_000616.5 | NP_000607.1 |
| | | | | NM_001195014.3 | NP001181943.1 |
| | | | | NM_001195015.3 | NP_001181944.1 |
| | | | | NM_001195016.3 | NP001181945.1 |
| | | | | NM_001195017.3 | NP001181946.1 |
| | | | | NM_001382705.1 | NP001369634.1 |
| | | | | NM_001382706.1 | NP001369635.1 |
| | | | | NM_001382707.1 | NP001369636.1 |
| | | | | NM_001382714.1 | NP_001369643.1 |
| CD8a | 1706 | ENSG00000153563 | P01732 | NM_001145873.1 | NP_001139345.1 |
| | | | | NM_001382698.1 | NP_001369627.1 |
| | | | | NM_001768.7 | NP_001759.3 |
| | | | | NM_171827.4 | NP_741969.1 |
| CD8b | 1707 | ENSG00000172116 | P10966 | NM_001178100.2 | NP001171571.1 |
| | | | | NM_004931.5 | NP_004922.1 |
| | | | | NM_172101.5 | NP_742099.1 |
| | | | | NM_172102.5 | NP_742100.1 |
| | | | | NM_172213.5 | NP_757362.1 |

Amino acid and nucleotide sequences corresponding to further variants and homologs of the above genes, as well as genes found in other species, may be found in similar publicly accessible databases or by identifying sequences showing homology to the above human sequences.

The present inventors have found that peptides derived from COVID-19 virus spike protein and envelope protein induce cardiac inflammation and an increase in the level of cMet-positive T cells. The sequences of the peptides, and corresponding sequence identifiers, are shown in the following table:

| Peptide | Source | | SEQ ID |
|---|---|---|---|
| name | protein | Sequence | NO |
| PA01 | envelope | MYSFVSEETGTLIVNSV | 1 |
| P87 | spike | LLHAPATVCGPKKST | 2 |
| PS556 | spike | NKKFLPFQQFGRDIA | 3 |
| P177 | spike | PHGVVFLHVTYVPAQ | 4 |

The sequence of COVID-19 virus envelope protein is SEQ ID NO 5 herein. The sequence of COVID-19 virus spike protein is SEQ ID NO 6 herein.

Determining a Level of cMet-Positive T Cells

The methods of the invention comprise a step of determining a level of cMet-positive T cells in a sample obtained from the subject.

“Determining a level of cMet-positive T cells” means measuring—either quantitatively or semi-quantitatively—the amount of cMet-positive T cells in a sample from the subject. Typically, the determination will reveal the absolute level of cMet-positive T cells in a sample from a subject, or the level of a cMet-positive T cells relative to the level of the reference value.

The level of cMet-positive T cells may be determined more than once in a given sample, for example for the purpose of statistical calculations. Alternatively, or in addition, a level may be determined one or more times in more than one sample obtained from the subject.

In some embodiments, “determining a level of cMet-positive T cells” means determining an absolute or approximate number of cMet-positive T cells. Accordingly, in such embodiments, the reference value is also an absolute number of cMet-positive T cells. In other words, in such embodiments, “comparing the level of cMet-positive T cells in the sample to a reference value” means comparing the absolute or approximate number of cMet-positive T cells determined to be in the sample to an absolute number of cMet-positive T cells that is the reference value.

In some embodiments, “determining a level of cMet-positive T cells” means determining a proportion of cMet-positive T cells. In some embodiments, the proportion of cMet-positive T cells is the proportion of T cells in the sample that express cMet. In some embodiments, the proportion of cMet-positive T cells is the proportion of all mononuclear cells in the sample that express cMet. In some embodiments, the proportion of cMet-positive T cells is the proportion of all cells in the sample that express cMet. Accordingly, in such embodiments, the reference value is also a proportion of cMet-positive T cells. In other words, in such embodiments, “comparing the level of cMet-positive T cells in the sample to a reference value” means comparing the proportion of cMet-positive T cells determined to be in the sample to a proportion of cMet-positive T cells that is the reference value. In some embodiments, the reference value is a proportion of T cells that express cMet. In some embodiments, the reference value is a proportion of mononuclear cells that express cMet. In some embodiments, the reference value is a proportion of all cells that express cMet. In any of these embodiments, the proportion of cMet-positive T cells may be expressed as a percentage.

cMet-positive T cells may be detected using flow cytometry. Flow cytometry is widely used in the art and essentially entails characterising features of cells in a sample by detecting fluorescence (see for example Mckinnon Curr Protoc Immunol 2019 120:5.1.1-5.1.11). In a typical example of a flow cytometry protocol, a sample of cells is stained with a primary antibody against a specific cell surface protein, and then with a secondary antibody conjugated to a fluorescent moiety. The sample is then passed through a flow cytometer, wherein the cells flow individually past excitatory lasers and a fluorescence detector. In this manner, the number of fluorescent cells (and so the number of cells expressing the protein of interest) can be counted, and sorted if required. Antibodies and other reagents for use in flow cytometry to detect cMet-positive T cells are known in the art and commercially available (see for example Komarowska et al. Immunity 2015 42, 1087-1099). Accordingly, in some embodiments of the methods of the invention, the level of cMet-positive T cells is determined by flow cytometry. Flow cytometry may also be referred to as fluorescence-activated cell sorting (FACS). Determination of the level of cMet-positive T cells by flow cytometry has the particular advantages of being faster than existing methods of diagnosis, with the time required to stain a whole blood sample from a subject for flow cytometry being less than 1 hour, such as about 52 minutes or as low as 30 minutes. In some embodiments, the level of cMet-positive T cells is determined using flow cytometry by staining the cells with labelled antibodies to cMet. In some embodiments, the level of cMet-positive T cells is determined using flow cytometry by staining the cells with labelled cMet ligand, preferably labelled HGF.

In some embodiments of the invention, the step of determining a level of cMet-positive T cells is carried out:
(a) before the onset of a cardiac inflammatory condition in the subject; and/or
(b) whilst the subject is showing symptoms of a cardiac inflammatory condition; and/or
(c) during and/or after the use of a treatment, therapy or prophylaxis for the cardiac inflammatory condition; and/or
(d) whilst the subject is showing symptoms of COVID-19; and/or
(e) during and/or after the subject has been treated with an anti-COVID-19 agent.

### Reference Value

In the methods of the present invention, the level of cMet-positive T cells in the sample is compared to a reference value. A “reference value” may include, but is not limited to, a value obtained from a reference subject (and samples obtained therefrom), or pre-determined absolute values.

Typically, a reference value is derived from a healthy subject, a subject known to be suffering from a particular condition or disease, or a subject who has recovered from a particular condition or disease. In the context of the present invention, the reference value may be derived from one or more of:
(a) a subject who has never suffered from a cardiac inflammatory condition;
(b) a subject who has recovered from a cardiac inflammatory condition;
(c) a subject who has never been infected with COVID-19 virus;
(d) a subject who has recovered from COVID-19;
(e) a subject who has not been vaccinated against COVID-19; and
(f) a subject who has been vaccinated against COVID-19 and did not develop a cardiac inflammatory condition.

The reference value may be, for example, a predetermined measurement of a level of cMet-positive T cells which is present in a sample from a normal subject, i.e. a subject who is not suffering from a cardiac inflammatory condition or any of (a) to (f) above. The reference value may, for example, be based on a mean or median level of cMet-positive T cells in a control population of subjects, e.g. 5, 10, 100, 1000 or more subjects (who may either be age- and/or gender-matched or unmatched to the test subject) who show no symptoms of a cardiac inflammatory condition.

The reference value may be determined using corresponding methods to the determination of level of cMet-positive T cells in the test sample, e.g. using one or more samples taken from a control population of subjects. For instance, in some embodiments levels of cMet-positive T cells in reference value samples may be determined in parallel assays to the test samples. In alternative embodiments, the reference value may have been previously determined, or may be calculated or extrapolated, without having to perform a corresponding determination on a reference value with respect to each test sample obtained.

In one embodiment, the reference value is derived from a subject suffering from a cardiac inflammatory condition. In one embodiment, the reference value is derived from the same subject, but at an earlier time. Thus, the invention may enable the status of a subject, such as disease progression in a subject, to be monitored over time. In particular, this embodiment finds utility when monitoring the response to therapy for the cardiac inflammatory condition over time.

In some embodiments, the reference value is a range of values. For example, it may be determined that healthy subjects present levels of cMet-positive T cells within a particular “healthy” range. Equally, subjects suffering from a cardiac inflammatory condition may present levels of a cMet-positive T cells within a particular “disease” range. Reference values, and in particular ranges of values may be optimised over time as more data are obtained and analysed.

### Comparing to Reference Value

In the diagnostic methods of the invention, the level of cMet-positive T cells in the sample compared to the reference value is indicative of the presence or absence of the cardiac inflammatory condition in the subject.

“Indicative of the presence or absence of the cardiac inflammatory condition” means the act of diagnosis of a cardiac inflammatory condition in a subject (i.e. positive diagnosis), or the act of ruling out a diagnosis of a cardiac inflammatory condition in a subject (i.e. negative diagnosis).

The level of cMet-positive T cells in the sample may be indicative of the presence or absence of the cardiac inflammatory condition. For example, the level of cMet-positive T cells may be merely suggestive, or may definitively denote the presence or absence of the cardiac inflammatory condition in a subject. Thus, in some embodiments, the level of cMet-positive T cells is suggestive of the presence or absence of the cardiac inflammatory condition. In other embodiments, the level of cMet-positive T cells denotes the presence or absence of the cardiac inflammatory condition.

In the methods of prediction of the invention, the level of cMet-positive T cells in the sample compared to the reference value is predictive of whether the subject will develop the cardiac inflammatory condition.

“Predictive of whether the subject will develop the cardiac inflammatory condition” means the act of predicting that the subject will develop the cardiac inflammatory condition (i.e. positive prediction), or the act of predicting that the subject will not develop the cardiac inflammatory condition (i.e. negative prediction).

The level of cMet-positive T cells in the sample may be predictive of the presence or absence of the cardiac inflammatory condition. For example, the level of cMet-positive T cells may be merely suggestive of a positive prediction, or may definitively denote that the subject will develop the cardiac inflammatory condition. Thus, in some embodiments, the level of cMet-positive T cells is suggestive that the subject will develop the cardiac inflammatory condition. In other embodiments, the level of cMet-positive T cells denotes that the subject will develop the cardiac inflammatory condition.

In some embodiments, an increased level of cMet-positive T cells in the sample compared to the reference value is indicative of the presence of the cardiac inflammatory condition in the subject. Thus, in some embodiments, a higher absolute number of cMet-positive T cells in the sample compared to the number of cMet-positive T cells that is the reference value is indicative of the presence of the cardiac condition in the subject. In some embodiments, a higher approximate number of cMet-positive T cells in the sample compared to the number of cMet-positive T cells that is the reference value is indicative of the presence of the cardiac condition in the subject. In some embodiments, a higher proportion of cMet-positive T cells in the sample compared to the reference value is indicative of the presence of the cardiac condition in the subject.

For example, a level of cMet-positive T cells in the sample of more than 1% of the total T cells in the sample may be indicative of the presence of the cardiac inflammatory condition. In this case, the reference value is 1% of the total T cells in sample.

In some embodiments, the level of cMet-positive T cells in the sample differs by at least 1%, at least 5%, at least 10%, at least 15%, at least 20%, at least 30%, at least 50%, at least 75% or at least 100% compared to the reference value. In some embodiments, the level of cMet-positive T cells in the sample differs by at least 2-fold, for example at least 3-fold, 4-fold, 5-fold, 6-fold, 8-fold or 10-fold compared to the reference value. Preferably, the level of cMet-positive T cells in the sample differs by at least 2-fold compared to the reference value.

In some embodiments, an unchanged level of cMet-positive T cells is indicative of absence of the cardiac inflammatory condition in the subject. By “unchanged level of cMet positive T cells” it is meant that the relative or absolute level of the cMet positive T cells is of substantially the same value compared to a reference value. In some embodiments, an unchanged level means a level which differs by less than 2-fold, such as less than 1.5-fold, compared to the reference value.

In some embodiments, the reference value is 0 or substantially 0. In other words, in some embodiments the reference value is that there are no, or substantially no, cMet-positive T cells. Thus, in some embodiments, the mere presence of cMet-positive T cells in the sample is indicative of the presence in the subject of the cardiac inflammatory condition. Likewise, in some embodiments, the presence of cMet-positive T cells in the sample predicts that the subject will develop the cardiac inflammatory condition.

As will be apparent to the person skilled in the art, the actual determination of whether a level of cMet-positive T cells is substantially increased, decreased or unchanged compared to a reference value may depend on the outcome of one or more statistical analyses, all of which are known and are routine to the person skilled in the art.

### Sample

The sample is not returned to the subject after the method of diagnosis or prediction has been carried out. The sample may also be referred to as “the test sample” herein. The sample may be or may be derived from an ex vivo sample.

In preferred embodiments, the sample obtained from the subject is whole blood or a fraction of whole blood. Preferably, the sample is peripheral whole blood. Peripheral whole blood is the circulating pool of blood, as opposed to that sequestered in the lymphatic system or organs. In some embodiments, the sample is processed prior to being subjected to the method of diagnosis or prediction of the invention. For example, peripheral blood mononuclear cells (PBMCs) may be isolated from peripheral blood obtained from a subject, and the PBMCs subjected to the method of diagnosis or prediction of the invention. Accordingly, in some embodiments, the sample is PBMCs obtained from the subject. PBMCs can be isolated from peripheral blood by density gradient centrifugation, as known in the art (Böyum Scand J Clin Lab Invest Suppl 1968 97:77-89).

Combination with Further Diagnostic Techniques

The diagnostic and predictive methods of the invention may be used in combination with existing methods for diagnosis of cardiac inflammatory conditions. Such existing methods to diagnose cardiac inflammation include electrocardiogram (detecting the pattern of electrical activity of the heart), echocardiogram (detecting sounds waves from the beating heart), chest X-ray (to assess heart morphology and presence of fluid in or around the heart), cardiac MRI (to assess heart morphology), cardiac catheterisation and heart muscle biopsy, and blood tests to check for proteins typically elevated in cardiac inflammation due to cardiac damage, such as cardiac troponin I (cTnI), cardiac troponin T (cTnT), b-type natriuretic peptide (BNP) and N-terminal proBNP (NT-proBNP). The person skilled in the art is familiar with, and can readily carry out, these existing diagnostic methods (see for example Siripanthong et al. Heart Rhythm 2020 17 (9): 1463-1471).

Thus, in some embodiments, the methods of the invention comprise an additional step of diagnosing the cardiac inflammatory condition in the subject using one or more diagnostic methods selected from the following: electrocardiogram, echocardiogram, chest X-ray, cardiac magnetic resonance imaging (MRI), cardiac catheterisation, heart muscle biopsy, determining a level of cardiac troponin I (cTnI) in a blood sample from the subject, determining a level of cardiac troponin T (cTnT) in a blood sample from the subject, determining a level of b-type natriuretic peptide (BNP) in a blood sample from the subject, and determining a level of N-terminal proBNP (NTproBNP) in a blood sample from the subject. The level of cTnI in the sample may be compared to a cTnI reference value, wherein the level of cTnI in the sample compared to the cTnI reference value is indicative of the presence or absence of the cardiac inflammatory condition in the subject. The level of cTnT in the sample may be compared to a cTnT reference value, wherein the level of cTnT in the sample compared to the cTnT reference value is indicative of the presence or absence of the cardiac inflammatory condition in the subject. The level of BNP in the sample may be compared to a BNP reference value, wherein the level of BNP in the sample compared to the BNP reference value is indicative of the presence or absence of the cardiac inflammatory condition in the subject. The level of NTproBNP in the sample may be compared to a NTproBNP reference value, wherein the level of NTproBNP in the sample compared to the NTproBNP reference value is indicative of the presence or absence of the cardiac inflammatory condition in the subject.

### Therapy of Cardiac Inflammatory Condition

The present invention also provides methods of treating a cardiac inflammatory condition in a subject. In particular, the invention provides a method of treating a subject suffering from a cardiac inflammatory condition, the method comprising the steps of:
(a) diagnosing a cardiac inflammatory condition using the method for diagnosing a cardiac inflammatory condition of the invention; and
(b) administering to the patient a therapeutically effective amount of a treatment, therapy or prophylaxis for a cardiac inflammatory condition.

Thus, the invention provides a method of treating a subject suffering from a cardiac inflammatory condition, the method comprising the steps of:
(a) diagnosing a cardiac inflammatory condition in a subject, wherein the subject is suffering from COVID-19, and/or has been vaccinated against COVID-19 within 30 days prior to carrying out said diagnosis, the diagnosis comprising the steps of:
   (i) determining a level of cMet-positive T cells in a sample obtained from the subject; and
   (ii) comparing the level of cMet-positive T cells in the sample to a reference value, wherein the level of cMet-positive T cells in the sample compared to the reference value is indicative of the presence or absence of the cardiac inflammatory condition in the subject; and
(b) administering to the patient a therapeutically effective amount of a treatment, therapy or prophylaxis for a cardiac inflammatory condition.

The method of treating the cardiac inflammatory condition in a subject may further comprise a step (c) repeating step (a) after administration of the treatment, therapy or prophylaxis. If the cardiac inflammatory condition is still present (as compared to that determined in step (a)), the method for treating the cardiac inflammatory condition in a subject may further comprise a step (d) administering a therapeutically effective amount of an alternative treatment, therapy or prophylaxis for a cardiac inflammatory condition to the subject, wherein the alternative treatment, therapy or prophylaxis differs from the treatment, therapy or prophylaxis administered in step (b).

The invention also provides a therapeutically effective amount of a treatment, therapy or prophylaxis for a cardiac inflammatory condition for use in a method of treating a subject suffering from a cardiac inflammatory condition, wherein the patient has been diagnosed with a cardiac inflammatory condition using the method for diagnosing a cardiac inflammatory condition of the invention.

Thus, the invention also provides a therapeutically effective amount of a treatment, therapy or prophylaxis for a cardiac inflammatory condition for use in a method of treating a subject suffering from a cardiac inflammatory condition, wherein the patient has been diagnosed with a cardiac inflammatory condition using a method for diagnosing a cardiac inflammatory condition in a subject, wherein the subject
(i) is suffering from COVID-19, and/or
(ii) has been vaccinated against COVID-19 within 30 days prior to carrying out said method of diagnosis,
   the method comprising the steps of:
(a) determining a level of cMet-positive T cells in a sample obtained from the subject; and
(b) comparing the level of cMet-positive T cells in the sample to a reference value, wherein the level of cMet-positive T cells in the sample compared to the reference value is indicative of the presence or absence of the cardiac inflammatory condition in the subject.

The treatment, therapy or prophylaxis for a cardiac inflammatory condition may be any suitable agent that can treat or alleviate the signs and/or symptoms of the cardiac inflammatory condition. The treatment, therapy or prophylaxis can be one or more treatment, therapy or prophylaxis that may be administered over a time course and/or simultaneously or at different times.

In some embodiments, the treatment, therapy or prophylaxis for a cardiac inflammatory condition is one or more of: a glucokinase activator, an antiviral drug, an inhibitor of effector T cells, an activator of regulatory T cells, a steroidal or non-steroidal immunosuppressive drug such as an anti-inflammatory biologic, colchicine, a beta-blocker, an angiotensin converting enzyme (ACE) inhibitor, an angiotensin receptor blocker (ARB), a mineralocorticoid antagonist, a neprolysin inhibitor, a sodium-glucose transport protein 2 (SGLT2) inhibitor, a diuretic, an antibiotic or intravenous immunoglobulin (IVIG).

An example of a glucokinase activator for use in the invention is piragliatin. Examples of antiviral drugs for use in the invention include Lagevrio (molnupiravir) and Paxlovid (PF-07321332/ritonavir). Example of inhibitors of effector T cells for use in the invention include methylprednisolone and tocilizumab (anti-IL-6). Examples of activators of regulatory T cells for use in the invention include low-dose rapamycin and Tregalizumab (a non-depleting IgG1 mAb that binds to a unique epitope of CD4 and selectively induces Treg activation). Examples of immune suppressive drugs for use in the invention include cyclosporin, tacrolimus, azathioprine and mycophenolate mofetil. Examples of steroid drugs for use in the invention include prednisolone, methylprednisolone and hydrocortisone. Examples of non-steroidal anti-inflammatory drugs for use in the invention include aspirin and ibuprofen. Examples of anti-inflammatory biologics include monoclonal antibodies directed to components of the immune system including membrane-bound targets and receptors, extracellular proteins and cytokines. Examples of beta-blockers for use in the invention include acebutolol and propranolol. Examples of ACE inhibitors for use in the invention include benazepril and captopril. Examples of ARBs for use in the invention include irbesartan, valsartan, losartan and candesartan. Examples of mineralocorticoid antagonists for use in the invention include spironolactone and eplerenone. Examples of neprolysin inhibitors for use in the invention include sacubitril combined with the ARB inhibitor valsartan in the formulation known as Entresto. Examples of SGLT2 inhibitors include canagliflozin, dapagliflozin and empagliflozin. An example of a diuretic for use in the invention is furosemide. An example of an antibiotic for use in the invention is clarithromycin.

Suitable dosage amounts and regimens of the treatment, therapy or prophylaxis for a cardiac inflammatory condition to be used in conjunction with the present invention may be adequately determined by the person skilled in the art. For example, a treatment, therapy or prophylaxis may be formulated and administered to a subject in any suitable composition for the treatment of a cardiac inflammatory condition. In particular embodiments, an effective amount of a treatment, therapy or prophylaxis is administered to the subject. In this context, the term “effective amount” or “therapeutically effective amount” means an amount effective, at dosages and for periods of time necessary to achieve the desired result, for example, to treat the cardiac inflammatory conditions.

The treatment, therapy or prophylaxis may be administered to a subject using a variety of techniques. For example, the treatment, therapy or prophylaxis may be administered systemically, which includes by injection including intramuscularly or intravenously, orally, sublingually, transdermally, subcutaneously, internasally. Alternatively, the treatment, therapy or prophylaxis may be administered directly at a site affected by the cardiac inflammatory condition.

The concentration and amount of the treatment, therapy or prophylaxis to be administered will typically vary, depending on, for example, the severity of the cardiac inflammatory condition, the type of treatment, therapy or prophylaxis that is administered, the mode of administration, the age and health of the subject, and the like.

The treatment, therapy or prophylaxis may be an agent formulated in a pharmaceutical composition together with a pharmaceutically acceptable carrier, vehicle, excipient or diluent. The compositions may routinely contain pharmaceutically acceptable concentrations of salt, buffering agents, preservatives and various compatible carriers. For instance the agent may be formulated in a physiological buffer solution.

The proportion and identity of the pharmaceutically acceptable carrier, vehicle, excipient or diluent may be determined by the chosen route of administration, compatibility with live cells, and standard pharmaceutical practice. Generally, the pharmaceutical composition will be formulated with components that will not significantly impair the biological properties of the agent. Suitable carriers, vehicles, excipients and diluents are described, for example, in Remington's Pharmaceutical Sciences (Remington's Pharmaceutical Sciences, Mack Publishing Company, Easton, Pa., USA 1985).

This disclosure is not limited by the exemplary methods and materials disclosed herein, and any methods and materials similar or equivalent to those described herein can be used in the practice or testing of embodiments of this disclosure. Numeric ranges are inclusive of the numbers defining the range. Unless otherwise indicated, amino acid sequences are written left to right in amino to carboxy orientation, respectively.

It must be noted that as used herein and in the appended claims, the singular forms “a”, “an”, and “the” include plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps. The terms “comprising”, “comprises” and “comprised of” also include the term “consisting of”.

The publications discussed herein are provided solely for their disclosure prior to the filing date of the present application. Nothing herein is to be construed as an admission that such publications constitute prior art to the claims appended hereto. All publications mentioned in the above specification are herein incorporated by reference.

Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in immunology, molecular biology, virology, cardiac pathology or related fields are intended to be within the scope of the following claims.

The invention will now be further described by way of Examples, which are meant to serve to assist one of ordinary skill in the art in carrying out the invention and are not intended in any way to limit the scope of the invention.

### EXAMPLES

### Example 1—Identification of COVID Peptides

The sequences of the SARS-COV-2 coronavirus envelope and spike proteins were compared to human protein sequences to identify regions of sequence identity. This comparison identified four amino acid sequences (one within the envelope protein sequence and three within the spike protein sequence) that have identity to sequences in human proteins. The four amino acid sequences (“COVID peptides”) are shown in Table 1.

TABLE 1

| Peptide | Source | | SEQ ID |
|---|---|---|---|
| name | protein | Sequence | NO |
| PA01 | envelope | MYSFVSEETGTLIVNSV | 1 |
| P87 | spike | LLHAPATVCGPKKST | 2 |
| PS556 | spike | NKKFLPFQQFGRDIA | 3 |
| P177 | spike | PHGVVFLHVTYVPAQ | 4 |

The full length sequences of SARS-COV-2 coronavirus envelope protein (SEQ ID NO 5) and spike protein (SEQ ID NO 6) are shown below, with the location of the peptides shown by shading.

SARS-CoV-2 envelope protein
(SEQ ID NO 5)

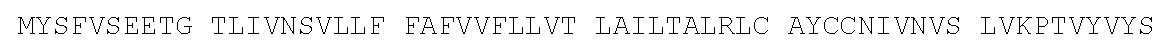

RVKNLNSSEG VPDLLV
SARS-CoV-2 spike protein
(SEQ ID NO 6)
MFVFLVLLPL VSSQCVNLTT RTQLPPAYTN SFTRGVYYPD KVFRSSVLHS TQDLFLPFFS
NVTWFHAIHV SGTNGTKRFD NPVLPFNDGV YFASTEKSNI IRGWIFGTTL DSKTQSLLIV
NNATNVVIKV CEFQFCNDPF LGVYYHKNNK SWMESEFRVY SSANNCTFEY VSQPFLMDLE
GKQGNFKNLR EFVFKNIDGY FKIYSKHTPI NLVRDLPQGF SALEPLVDLP IGINITRFQT
LLALHRSYLT PGDSSSGWTA GAAAYYVGYL QPRTFLLKYN ENGTITDAVD CALDPLSETK
CTLKSFTVEK GIYQTSNFRV QPTESIVRFP NITNLCPFGE VFNATRFASV YAWNRKRISN
CVADYSVLYN SASFSTFKCY GVSPTKLNDL CFTNVYADSF VIRGDEVRQI APGQTGKIAD
YNYKLPDDFT GCVIAWNSNN LDSKVGGNYN YLYRLFRKSN LKPFERDIST EIYQAGSTPC

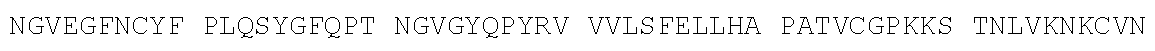

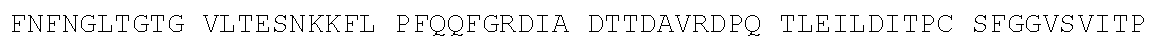

GTNTSNQVAV LYQNVNCTEV PVAIHADQLT PTWRVYSTGS NVFQTRAGCL IGAEHVNNSY
ECDIPIGAGI CASYQTQTNS PSRAGSVASQ SIIAYTMSLG AENSVAYSNN SIAIPTNFTI
SVTTEILPVS MTKTSVDCTM YICGDSTECS NLLLQYGSFC TQLNRALTGI AVEQDKNTQE
VFAQVKQIYK TPPIKDFGGF NFSQILPDPS KPSKRSFIED LLFNKVTLAD AGFIKQYGDC
LGDIAARDLI CAQKFNGLTV LPPLLTDEMI AQYTSALLAG TITSGWTFGA GPALQIPFAM
QMAYRFNGIG VTQNVLYENQ KLIANQFNSA IGKIQDSLSS TPSALGKLQD VVNQNAQALN
TLVKQLSSNF GAISSVLNDI LSRLDKPEAE VQIDRLITGR LQSLQTYVTQ QLTRAAEIRA

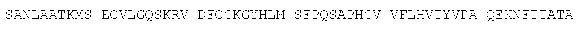

ICHDGKAHFP REGVFVSNGT HWFVTQRNFY EPQIITTDNT FVSGNCDVVI GIVNNTVYDP
LQPELDSFKE ELDKYFKNHT SPDVDLGDIS GINASVVNIQ KEIDRLNEVA KNLNESLIDL
QELGKYEQGS GYIPEAPRDG QAYVRKDGEW VLLSTFLGRS LEVLFQGPGS LPETGGGSDY
KDDDDKGGGG SGGGGSGGGG SGGGGSGGGG SHHHHHH

The human proteins with which the COVID peptides share sequence are shown in Tables 2 to 5, along with alignments to parts of their sequences.

TABLE 2

| Peptide/ | | | SEQ |
|---|---|---|---|
| human | Sequence | | ID |
| protein | location | Sequence | NO |
| PA01 | — | MYSFVSEETGTLIVNSV | 1 |
| S-formyl- | 120-127 | MYSYVTEE | 7 |
| glutathione | | | |
| hydrolase | | | |
| PTPN13 | 1496-1502 | YSFVTEE | 8 |
| protein | 744-749 | VSEETG | 9 |
| O-mannosyl- | | | |
| transferase | | | |
| TMTC1 | | | |
| LRRC8E | 342-352 | YSFRSVREETG | 10 |
| PRKAB2 | 193-200 | MYAFRSEE | 11 |
| ZFP106 | 874-883 | YSFFSEEGTG | 12 |

TABLE 3

| Peptide/ | Sequence | | SEQ |
|---|---|---|---|
| human protein | location | Sequence | ID NO |
| P87 | — | LLHAPATVCGPKKST | 2 |
| KCNC2 | 123-132 | LHCPADVCGP | 13 |
| SHANK2 | 284-292 | LLHEHATVC | 14 |

TABLE 4

| Peptide/ | | | SEQ |
|---|---|---|---|
| human | Sequence | | ID |
| protein | location | Sequence | NO |
| Ps556 | — | NKKF---LPFQQFGRDIA | 3 |
| serpin B4 | 4-19 | LSEANTKFMFDL-FQQF | 15 |
| (isoform 2) | | | |
| KCNB1 | 279-290 | LTESN-KSV--LQFQ | 16 |

TABLE 5

| | | | SEQ |
|---|---|---|---|
| Peptide/ | Sequence | | ID |
| human protein | location | Sequence | NO |
| P177 | — | PHGVVFLHVTYVPAQ | 4 |
| HEAT repeat- | 172-180 | GVVFPHVSY | 17 |
| containing | | | |
| protein 5B | | | |
| isoform | | | |
| N-acetylmuramoyl- | 406-416 | FLYVHHTYVPA | 18 |
| L-alanine | | | |
| amidase | | | |
| (isoform 1 | | | |
| precursor) | | | |
| myeloid cell | 102-108 | LNV--TYVP | 19 |
| surface antigen | | | |
| CD33 isoform 2 | | | |

### Example 2—Subcutaneous Immunisation of Mice with COVID Peptide Cocktail Including Different Adjuvants Induces Inflammation of the Heart

Mice were immunised subcutaneously with two doses, 1 week apart, of a cocktail of the four COVID peptides including either no adjuvant (control) or one of the following adjuvants: alum+MPLA, R848 (Resiquimod), or AS03+MPLA.

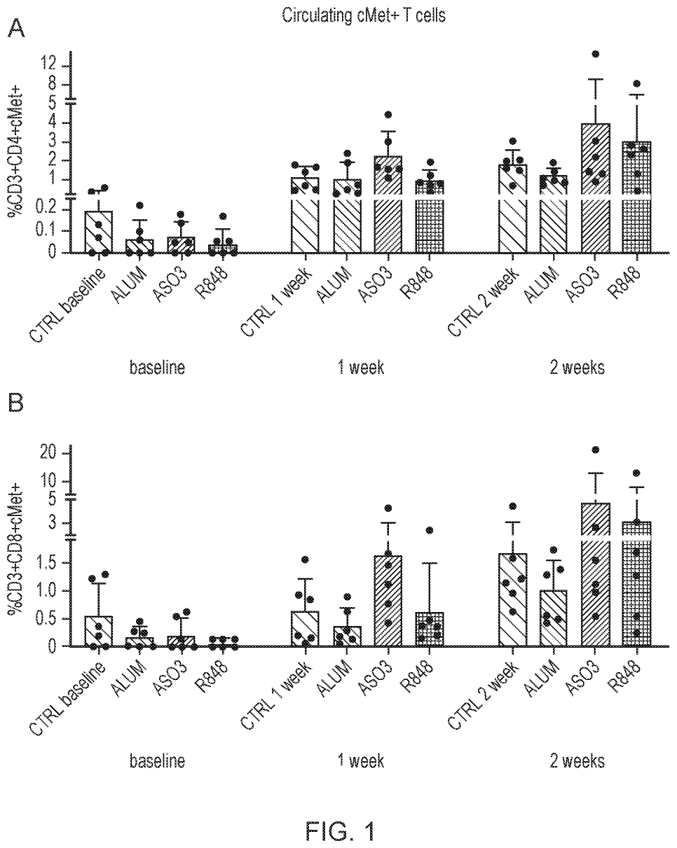

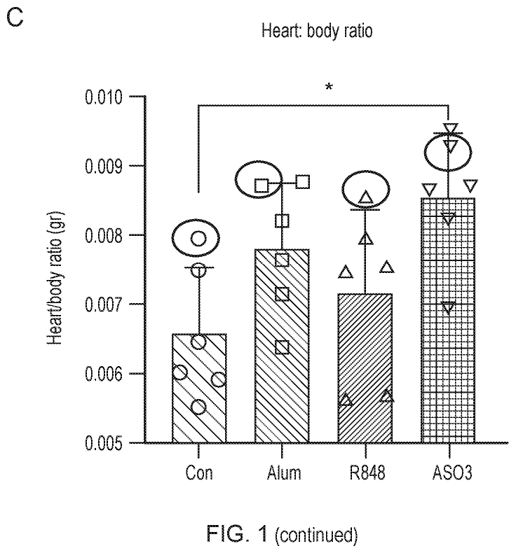

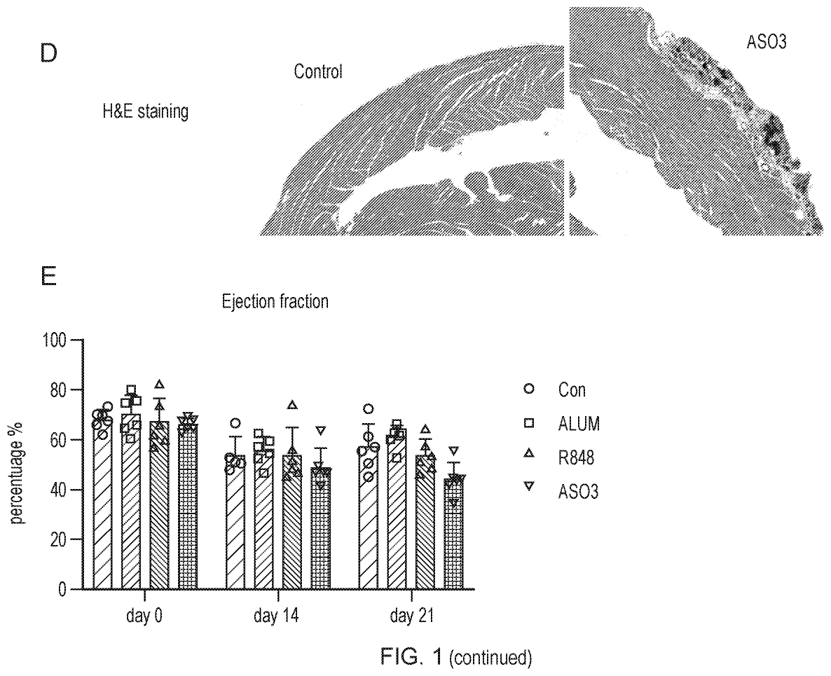

The levels of circulating CD3+CD4+ cMet+ T cells (FIG. 1A) and CD3+CD8+ cMet+ T cells (FIG. 1B) in the mice were measured. The level of CD3+CD4+ cMet+ T cells had increased markedly 1 week after the first immunisation and remained high 1 week after the second immunisation, regardless of whether adjuvant was included in the cocktail (see FIG. 1A). Similarly, the level of CD3+CD8+ cMet+ T cells increased by 1 week after the first immunisation and was even higher 1 week after the second immunisation (see FIG. 1B). The highest level of both CD3+CD4+ cMet+ and CD3+CD8+ cMet+ T cells was observed when AS03+MPLA adjuvant was included in the cocktail.

The heart weight to body weight ratio of the mice was measured (FIG. 1C). A higher heart:body weight ratio indicates a heavier heart, which may be due to inflammation. Mice immunised with peptide cocktail comprising AS03+MPLA adjuvant had a significantly higher heart:body weight ratio than mice treated with a peptide cocktail lacking adjuvant (control), indicating a higher degree of heart inflammation was induced by peptide cocktail comprising AS03+MPLA adjuvant.

FIG. 1D shows haematoxylin and eosin staining of a heart from a control mouse (immunised with peptide cocktail with no adjuvant) and a heart from a mouse immunised with peptide cocktail including AS03+MPLA adjuvant.

Heart ejection fraction (percentage of blood leaving the heart each time it contracts) of the mice was measured (FIG. 1E). Heart ejection fraction was reduced by 14 days, and even further reduced by 21 days, for all mice, indicating that immunisation with the peptide cocktail reduces heart function. The greatest reduction in heart ejection fraction was observed when the peptide cocktail included AS03+MPLA adjuvant.

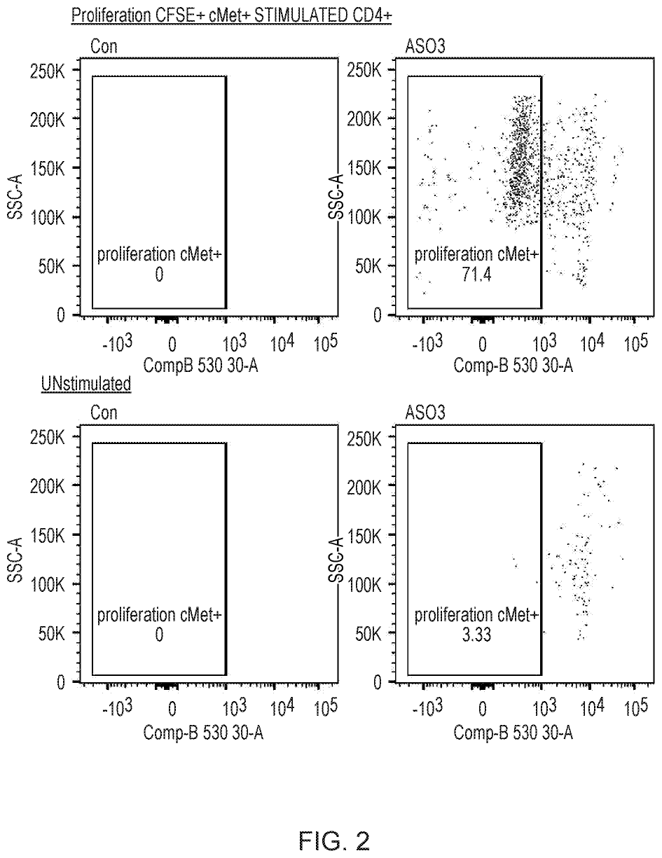

Proliferation of CD4+ cMet+ T cells upon immunisation of mice with peptide cocktail, with or without AS03+MPLA adjuvant, was compared to proliferation in non-immunised mice using fluorescence-activated cell sorting (FACS) (FIG. 2). The data show greatly increased proliferation of CD4+ cMet+ T cells in mice immunized with peptide cocktail including AS03+MPLA.

In summary, these data indicate that immunisation of mice with a cocktail of the COVID peptides induces inflammation of the heart, correlating with reduced heart function and an increase in circulating cMet+ T cells. The adjuvant AS03+MPLA particularly enhances the effect of administration of the peptide cocktail.

### Example 3—Intranasal Immunisation of Mice with COVID Peptide Cocktail Including AS03 Induces Inflammation of the Heart

To mimic infection with SARS-COV-2 coronavirus, mice were immunised via the intranasal route with two doses, 1 week apart, of a cocktail of the four COVID peptides and AS03+MPLA adjuvant. Control mice were immunised with AS03+MPLA alone.

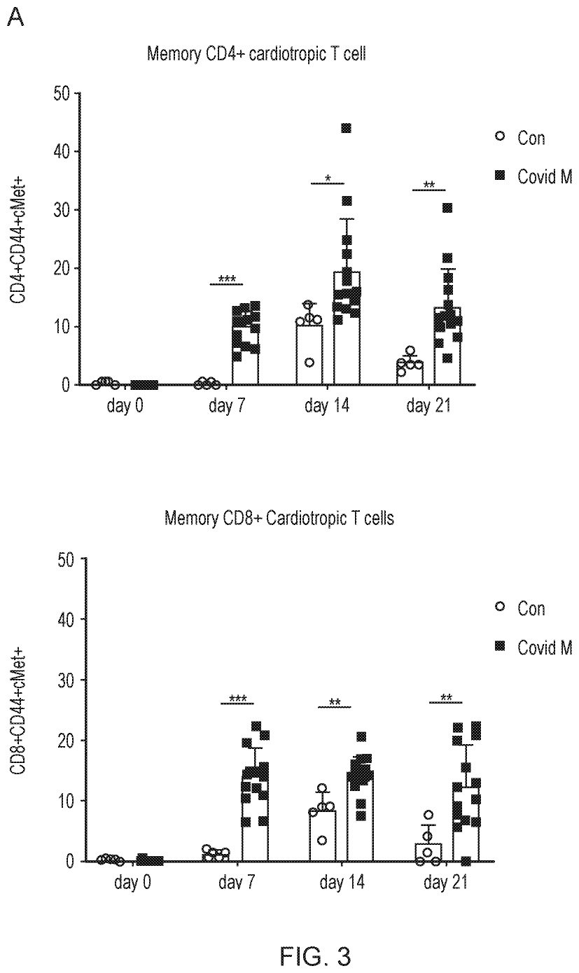

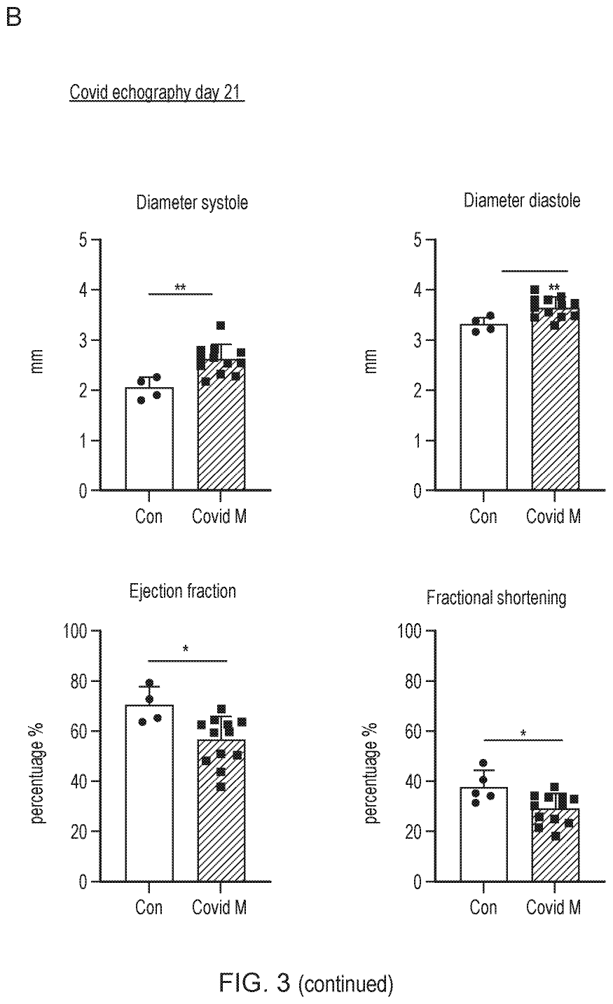

The levels of circulating CD4+ cMet+ T cells and CD8+ cMet+ T cells in the mice were measured (FIG. 3A). Levels of both types of T cells increased significantly in mice immunised with the peptide cocktail in comparison to control mice over the three weeks after the first immunisation.

Echocardiography was used to measure cardiac function three weeks after the first immunisation (FIG. 3B). Mice immunised with the peptide cocktail had increased heart diameter at systole (contracted heart) and diastole (relaxed heart), reduced ejection fraction (percentage of blood leaving the heart each time it contracts) and reduced fractional shortening (percentage change in left ventricle diameter during systole).

FIG. 3C shows haematoxylin and eosin staining of hearts from two mice immunised with peptide cocktail including AS03+MPLA adjuvant. Both hearts show characteristics of myocarditis.

Together, these data show that immunisation of mice via the intranasal route with a cocktail of the COVID peptides induces inflammation of the heart, correlating with reduced heart function and an increase in circulating cMet+ T cells.

### Example 4—Immunisation of Mice with Individual COVID Peptides Increases cMet+ T Cells

Mice were immunised with two doses, 1 week apart, of a single COVID peptide with AS03+MPLA adjuvant. Control mice were immunised with adjuvant alone.

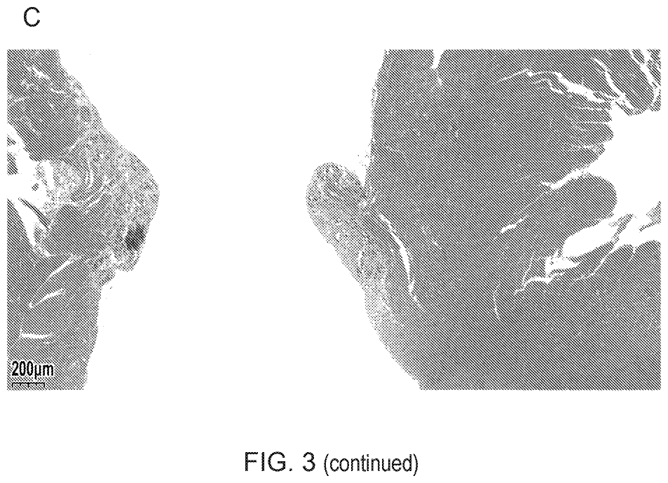

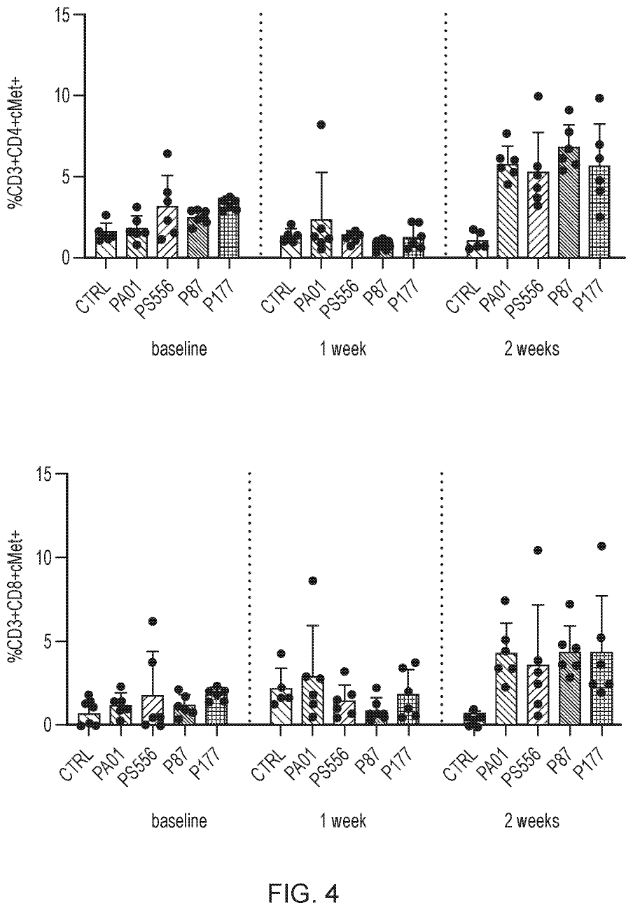

The levels of circulating CD3+CD4+ cMet+ T cells and CD3+CD8+ cMet+ T cells in the mice were measured (FIG. 4). The levels of both T cell types had increased markedly after two weeks from the first immunisation in mice immunised with any of the COVID peptides, but did not increase in control mice.

These data indicate that each of the identified COVID peptides alone can induce expansion of cardiotropic cMet+ T cells.

### Example 5—Flow Cytometry Results from Acute Myocarditis Patient

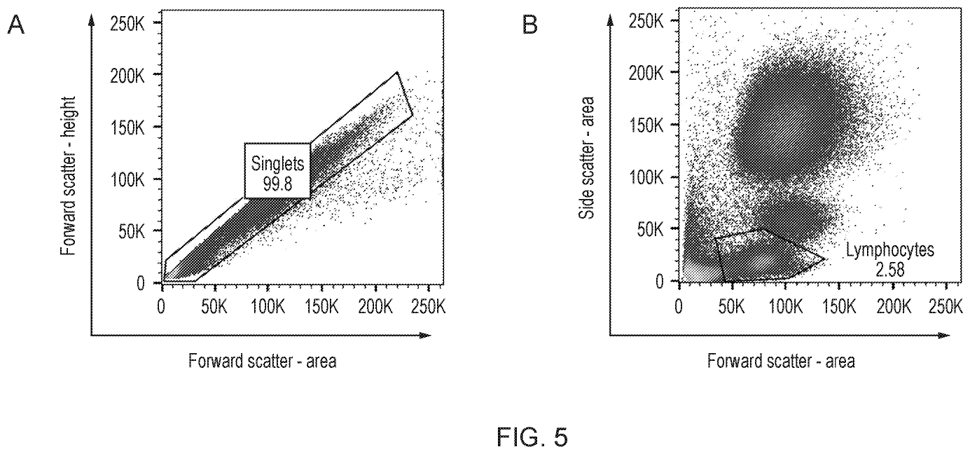

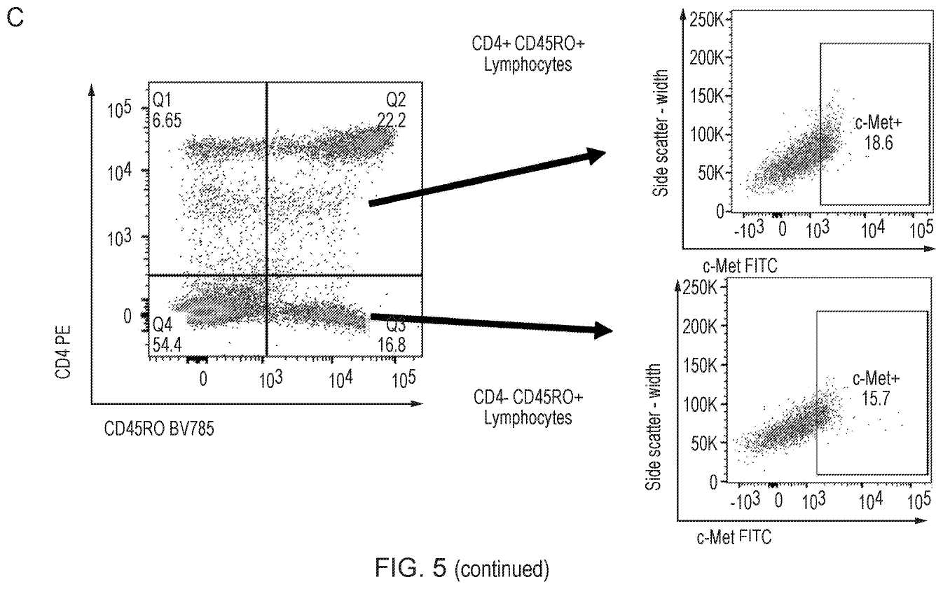

100 μL of whole peripheral blood from a patient with acute myocarditis was stained with anti-CD4, anti-CD45RO and anti-cMet antibodies in 52 minutes and the sample analysed by flow cytometry. FIG. 5 shows the gating strategy from data for single cells (A) and lymphocytes identified within the blood (B). Granulocyte and monocyte populations also seen. With gating on single cell lymphocytes, cMet expression of 15% to 18% is seen in this acute myocarditis patient with both CD4+CD45RO+ T cells, as well as CD4− (presumed to be CD8+) CD45RO+ T cells (C). Similar results were obtained in two other acute myocarditis patients.

1. A method for diagnosing a cardiac inflammatory condition in a subject, wherein the subject
(i) is infected with COVID-19 virus and/or suffering from one or more morbidity associated with COVID-19, and/or
(ii) has been vaccinated against COVID-19,
the method comprising the steps of:
(a) determining a level of cMet-positive T cells in a sample obtained from the subject; and
(b) comparing the level of cMet-positive T cells in the sample to a reference value, wherein the level of cMet-positive T cells in the sample compared to the reference value is indicative of the presence or absence of the cardiac inflammatory condition in the subject.

2. A method for predicting whether a subject will develop a cardiac inflammatory condition, wherein the subject
(i) is infected with COVID-19 virus and/or suffering from one or more morbidity associated with COVID-19, and/or
(ii) has been vaccinated against COVID-19,
the method comprising the steps of:
(a) determining a level of cMet-positive T cells in a sample obtained from the subject; and
(b) comparing the level of cMet-positive T cells in the sample to a reference value, wherein the level of cMet-positive T cells in the sample compared to the reference value is predictive of whether the subject will develop the cardiac inflammatory condition.

3. The method of claim 1 or claim 2, wherein the cardiac inflammatory condition is myocarditis.

4. The method of any one of claims 1 to 3, wherein the cardiac inflammatory condition is pericarditis.

5. The method of any one of claims 1 to 4, wherein the subject has been vaccinated against COVID-19 within 6 weeks prior to carrying out the method of diagnosis or method of prediction.

6. The method of any one of claims 1 to 5, wherein the subject is suffering from long COVID.

7. The method of any one of claims 1 to 6, wherein the subject has been vaccinated against COVID-19 using an mRNA vaccine.

8. The method of any one of claims 1 to 7, wherein the subject has been vaccinated against COVID-19 using BNT162b2 vaccine.

9. The method of any one of claims 1 to 7, wherein the subject has been vaccinated against COVID-19 using mRNA-1273 vaccine.

10. The method of any one of claims 1 to 9, wherein an increased level of cMet-positive T cells in the sample compared to the reference value is indicative of the presence of the cardiac inflammatory condition in the subject or predictive of whether the subject will develop the cardiac inflammatory condition.

11. The method of any one of claims 1 to 10, wherein the cMet-positive T cells are cardiotropic T cells.

12. The method of claim 11, wherein the cardiotropic cMet-positive T cells are virus-induced cardiotropic T cells.

13. The method of any one of claims 1 to 12, wherein the cMet-positive T cells are CCR4-positive T cells.

14. The method of any one of claims 1 to 13, wherein the cMet-positive T cells are CXCR3-positive T cells.

15. The method of any one of claims 1 to 14, wherein the cMet-positive T cells are CD4-positive T cells.

16. The method of any one of claims 1 to 15, wherein the cMet-positive T cells are CD8-positive T cells.

17. The method of any one of claims 1 to 16, wherein the subject is a male human.

18. The method any one of claims 1 to 17, wherein the subject is from 10 to 60 years of age.

19. A method of treating a subject suffering from a cardiac inflammatory condition, the method comprising the steps of:
(a) diagnosing a cardiac inflammatory condition using the method according to any one of claims 1 to 18;
(b) administering to the patient a therapeutically effective amount of a treatment, therapy or prophylaxis for a cardiac inflammatory condition.

20. A therapeutically effective amount of a treatment, therapy or prophylaxis for a cardiac inflammatory condition for use in a method of treating a patient suffering from a cardiac inflammatory condition, wherein the subject has been diagnosed with a cardiac inflammatory condition using the method as set forth in any one of claims 1 to 18.

21. The method of claim 19 or the treatment, therapy or prophylaxis for use of claim 20, wherein the treatment, therapy or prophylaxis for a cardiac inflammatory condition is one or more of: a glucokinase activator, an antiviral drug, an inhibitor of effector T cells, an activator of regulatory T cells, a steroidal or non-steroidal immunosuppressive drug such as an anti-inflammatory biologic, colchicine, a beta-blocker, an angiotensin converting enzyme (ACE) inhibitor, an angiotensin receptor blocker (ARB), a mineralocorticoid antagonist, a neprolysin inhibitor, a sodium-glucose transport protein 2 (SGLT2) inhibitor, a diuretic, an antibiotic or intravenous immunoglobulin (IVIG).
